(19) United States
(12) Patent Application Publication
(10) Pub. No.: US 2026/0256493 A1
(43) Pub. Date: Sep. 3, 2026

(54) SYSTEMS, DEVICES AND METHODS FOR MONITORING CURRENT IN A ROTATIONAL MEDICAL DEVICE AND LED MONITORING AND DISPLAY ARRAYS FOR ANNUNCIATING CURRENT AND PEAK VALUES OF A MONITORED VARIABLE

(71) Applicant: CARDIOVASCULAR SYSTEMS, INC., St. Paul, MN (US)

(72) Inventors: Matthew W. Tilstra, Rogers, MN (US); Nicholas J. Ellering, Crystal, MN (US)

(21) Appl. No.: 18/879,922

(22) PCT Filed: Mar. 21, 2023
(86) PCT No.: PCT/US2023/015761

§ 371 Date: Dec. 30, 2024

Related U.S. Application Data
(60) Provisional application No. 63/367,376, filed on Jun. 30, 2022.

(51) Int. Cl. A61B 17/3207; A61B 17/00

(52) U.S. Cl. CPC A61B 17/320758; A61B 2017/00075; A61B 2017/00119

(57) ABSTRACT

Various embodiments of the systems, methods and devices are provided to measure current by placing a current measuring device between an atherectomy device's pump and motor. In addition various embodiments of systems, methods and devices are provided with LED arrays on the atherectomy device handle, or external display, indicating real-time magnitudes of one or more monitored variables and peak value(s) of the monitored variable(s).

### CROSS-REFERENCE TO RELATED APPLICATIONS

This application claims the benefit of U.S. Provisional Application No. 63/367376, filed Jun. 30, 2022 and titled SYSTEMS, DEVICES AND METHODS FOR MONITORING CURRENT IN AN ATHERECTOMY DEVICE LOCATED BETWEEN PUMP AND MOTOR AND LEDS ANNUNCIATING CURRENT AND PEAK VALUES OF A MONITORED VARIABLE, the entire contents of which are incorporated herein by reference.

### STATEMENT REGARDING FEDERALLY SPONSORED RESEARCH OR DEVELOPMENT

None

### BACKGROUND OF THE INVENTION

### Field of the Invention

The invention relates to systems, devices and methods for rotating a drive shaft within a patient's vasculature. More specifically, systems, devices and methods for monitoring current in a rotational medical device and/or with an LED array annunciating real-time and peak values of a monitored variable.

### Description of the Related Art

Rotational medical devices are known in the art. For example, rotational atherectomy devices comprise a prime mover, e.g., an electric motor in operative rotational engagement with a rotational drive shaft. The rotational drive shaft comprises a tool for sanding and/or impacting a lesion located within a lumen of a subject blood vessel. Known rotational atherectomy devices monitor current at the motor. It would be advantageous to monitor current at another location within the system during rotational operation.

Moreover, known systems provide real-time feedback of a monitored motor variable an operator and may use a system comprising multiple LEDs for that purpose. See, e.g., U.S. Pat. No. 9,820,770 which employs LEDs to provide a visual output of a monitored variable, such as sensed current drawn at the atherectomy device motor, but does not provide a mechanism for displaying or annunciating the peak value of the monitored variable. Thus, the operator cannot visualize via the LEDs historical peak value data. This data is important to the operator when deciding if the procedure should be considered complete or if another treatment pass through the lesion is warranted and, if another treatment pass is desired, whether it should be executed at the same speed, higher speed or lower speed compared with previous passes.

Various embodiments of the present invention address the issues, among others, discussed above.

### BRIEF DESCRIPTION OF THE SEVERAL VIEWS OF THE DRAWINGS

FIG. 1A is a top view of an exemplary prior art atherectomy device handle.

FIG. 1B is a top view of an exemplary prior art atherectomy device with rotational drive shaft and rotational abrasive element.

FIG. 2 is a perspective cutaway view of one embodiment of the present invention.

FIG. 3A illustrates one graphic embodiment of the present invention.

FIG. 3B illustrates one monitoring and display embodiment for the values graphed in FIG. 3A.

FIG. 4A illustrates one graphic embodiment of the present invention.

FIG. 4B illustrates exemplary LEDs used for the values graphed in FIG. 4A.

FIG. 5A illustrates one graphic embodiment of the present invention.

FIG. 5B illustrates exemplary LEDs used for the values graphed in FIG. 5A.

FIG. 6 illustrates a top perspective view of one embodiment of the present invention.

FIG. 7A illustrates one graphic embodiment of the present invention.

FIG. 7B illustrates an exemplary monitor and display for the values graphed in FIG. 7A.

FIG. 8A illustrates one graphic monitor and display embodiment of the present invention.

FIG. 8B illustrates one graphic monitor and display embodiment of the present invention.

FIG. 9 provides a schematic diagram of an atherectomy device with exemplary current monitoring.

### DETAILED DESCRIPTION OF THE INVENTION

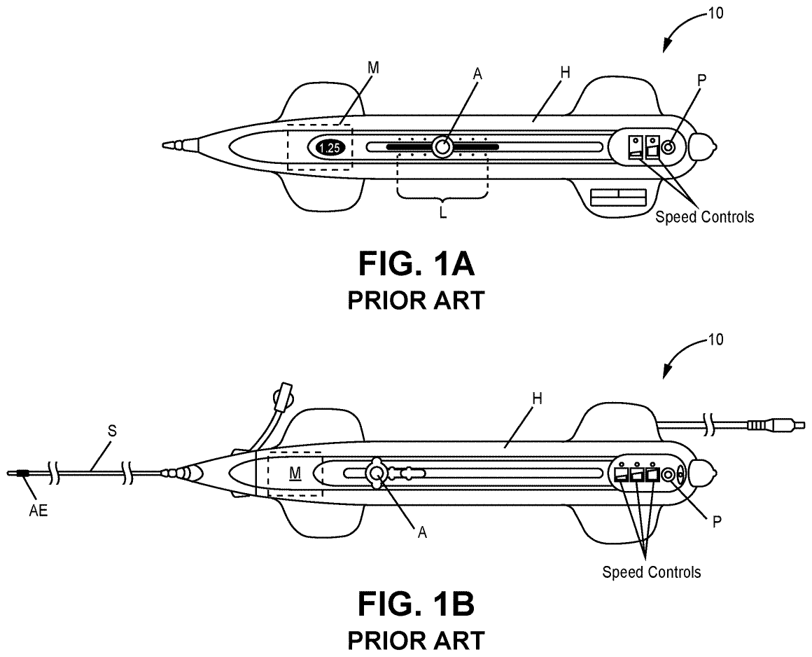

Various embodiments of the present invention comprise improvement through provision of visual status feedback to an operator of a rotational medical device, for example an orbital atherectomy device 10 (“OAD”) such as DIAMONDBACK 360® device marketed by Cardiovascular Systems, Inc., as shown and illustrated in FIGS. 1A and 1B. Such devices or systems comprise a prime mover, e.g., an electric motor M in operative rotational engagement with a rotational drive shaft S, wherein the electric motor M may be located within the handle H together with associated controls such as an advancement knob A for advancing or retracting the rotational drive shaft, and the attached abrasive element AE, axially. FIG. 1A best shows the relative location scale L along which advancement knob A moves to assist in tracking the location of the abrasive element within the patient's vasculature. Speed controls and a saline prime control P are also provided on the handle H. The rotational drive shaft S comprises a tool such as an abrasive element AE for sanding and/or impacting a lesion located within a lumen of a subject blood vessel.

The visual status feedback may comprise information about the device in real time and may include a current state of a monitored variable and/or may display a peak value, generally this will be the most current peak value, of the monitored variable, though other variables beyond current may be monitored. The monitored variable(s) may aid in determining whether a procedure requires an additional “pass” through a lesion with the abrasive element AE and/or whether a procedure is complete. The monitored variable(s) may also provide the operator with the required information needed to assess the relative axial or longitudinal location within or along the lesion that may require additional atherectomy with the abrasive element AE.

Accordingly, the monitored variable may comprise one or more of:
the rotational speed of the electric motor and/or the rotating rotational drive shaft;
the rate of change of the rotational speed of the electric motor and/or the rotational drive shaft during a rotational procedure;
the voltage of the electric motor during a rotational procedure;
the rate of change of the voltage of the electric motor;
the current generated by the electric motor during a rotational procedure;
the rate of change of the current generated by the electric motor during a rotational procedure;
the torque applied by the electric motor to the rotational drive shaft.

The visual status feedback may comprise one or more visual annunciators such as, without limitation, LEDs, including an LED array for monitoring and displaying magnitude(s) of the monitored variable(s). The LED monitor and display array may be multicolored in one embodiment and may be a single color in another embodiment. The LED monitor and display array may be provided on a separate display screen and/or a PC or a tablet that is in operative connection (wired or wireless) with the atherectomy device, e. g, a processor that executes programmed instructions to determine whether to actuate an LED. In other embodiments the LED monitor and display array may be integrated into or on a rotational medical device's handle.

Accordingly, certain exemplary embodiments described herein may comprise a small screen or display disposed in or on or integrated into an atherectomy device handle that displays a running graph of the monitored variable(s) value as well as displaying the most current peak value of the monitored variable(s).

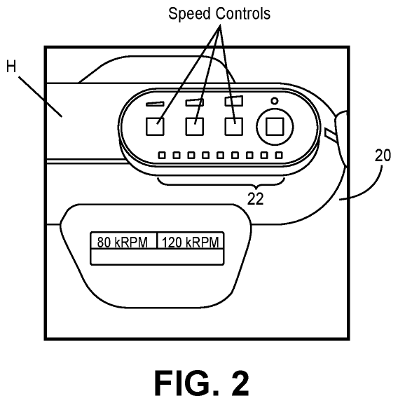

FIG. 2 illustrates one embodiment showing a distal portion of a rotational atherectomy device 20 wherein a LED monitor and display array 22 for monitoring a variable is incorporated on or into the device handle H. Other motor-driven rotational medical devices with handles H may also incorporate the LED monitor and display array 22 within the handle H. The LED monitor and display array 22 illustrated is exemplary and may comprise one or more of the LED monitor and display embodiments described herein.

The LED monitor and display array 22 of FIG. 2 provides a single line of LEDs (or other annunciators) that are lit when the monitored variable, e.g., current, reaches a predetermined magnitude that corresponds with a given LED within the single line of LEDs. The LED's in the array 22 are arranged to actuate or light as the magnitude of the monitored variable increases, such that one or more LEDs may be lit to allow the operator visual feedback of the monitored variable's magnitude. The LEDs of the array 22 are arranged in ascending order of magnitude of the monitored variable.

In some embodiments, the LEDs of the array 22 may change color as the magnitude of the monitored variable increases and reaches predetermined thresholds, e.g., lower magnitudes may be within a lower predetermined threshold range wherein the lit LEDs of the array 22 are all a first color, e.g., green. The next higher range of magnitudes may comprise LEDs within the array 22 that are a second color different from the first color, e.g., all yellow. The next higher range of magnitudes may comprise LEDs within the array 22 that are a third color different from the first and second colors, e.g., all red. As noted above, the highest magnitude monitored and displayed may be “sticky” in that the lit or actuated LED within the array 22 that corresponds with the highest magnitude reached during a procedure may remain lit or actuated or may flash to allow the operator to visualize the highest magnitude of the monitored variable during a given procedure. In other embodiments, the LEDs may not be “sticky”. In some embodiments, the LEDs of the LED monitoring and display array 22 may be arranged in a horizontal line as shown in FIG. 2, while in other embodiments, the LEDS of the array 22 may be arranged in a vertical line.

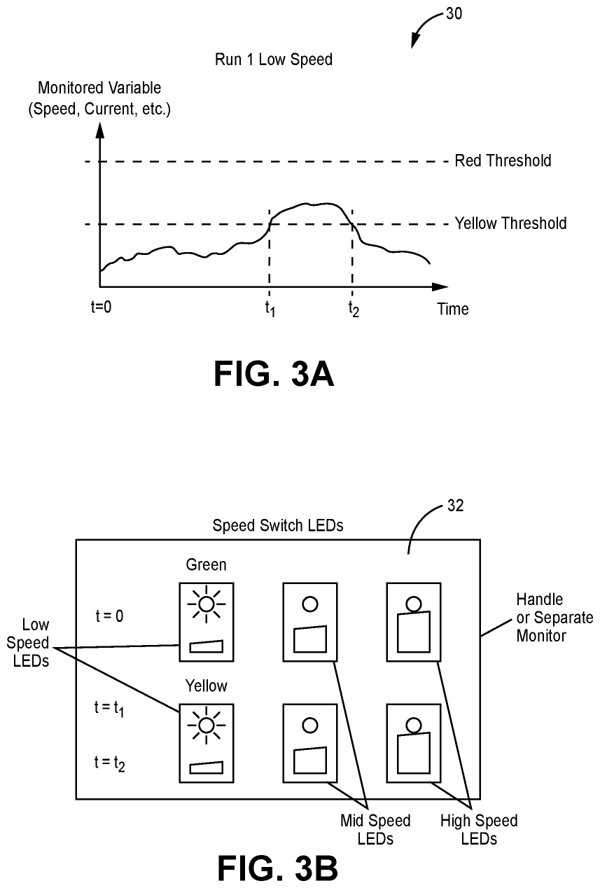

FIGS. 3A and 3B illustrate another embodiment of the present invention wherein, as in FIG. 3A, the monitored variable values are sensed and graphed over time by, e.g., a sensor that senses the data and communicates the sensed data to a processor comprising programmed instructions that are executed by a processor to generate a graphic 30. For example, the graphic 30 may comprise a first (yellow) threshold value and a second (red) threshold value, which is higher than the first (yellow) threshold value, are provided. The graphic of FIG. 3A may be displayed and/or may be calculated and graphed by the processor using the programmed instructions, which communicates the graphed data 30 to an operatively connected display for display and annunciation via an LED monitoring and display array 32 as in FIG. 3B. See also FIG. 10 and related discussion below. In other embodiments, the graphic 30 may be displayed on a display that is integrated into the handle H.

The LED monitoring and display array 32 of FIG. 3B is responsive to the generated graphic 30 and comprises a first low speed LED column, a second mid speed LED column and a third high speed LED column, the speed categories are merely exemplary and not intended to be limiting. The low, mid and high speeds are correspondent with the rotational speed of the exemplary atherectomy device motor and rotational drive shaft, wherein predetermined rotational speed windows are established, each predetermined rotational speed window may comprise its own set of thresholds. There may be, in certain embodiments, at least one threshold including but not limited to two or more thresholds. The two shown as “red” and “yellow” are exemplary and illustrative only. These data may be retained in the programmed instructions to be executed by a processor as discussed further infra in connection with FIG. 10.

As the monitored variables value remain below the first (yellow) threshold between time 0 and time 1 in FIG. 3A's graphic 30, the low speed LED associated with time between 0 and 1 may be lit as “green” in the array 32 of FIG. 3B, for example, to indicate that the monitored variable value is below all threshold(s).

As the monitored variable value rises above the first (yellow) threshold between time 1and time 2 in the graphic 30, the low speed LED associated with that time period may be lit as “yellow” in the array 32 to indicate that the lower or yellow threshold has been met and/or exceeded.

Because FIGS. 3A and 3B arc operating with the exemplary atherectomy device rotating within the low speed window, only the first column of LEDs (low speed) of the array 32 in FIG. 3B are actuated.

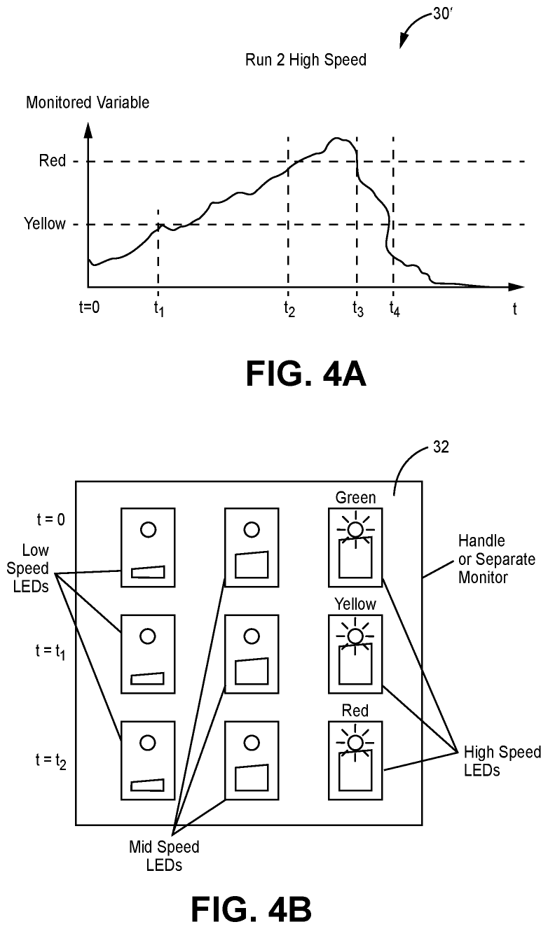

FIGS. 4A and 4B illustrate another exemplary atherectomy run with monitored variable value graphed over time 30′ and again with low, mid and high speed LED columns in the LED monitoring and display array 32, similar to that discussed in FIGS. 3A and 3B. In this example, the high speed LED column of the LED monitoring and display array 32 is actuated or lit because the rotational speed of the atherectomy device is above the high speed predetermined threshold.

The monitored variable's value remains below the first (yellow) threshold between times 0 and 1 (t₀-t₁) in graphic 30′ of FIG. 4A, so the high speed LED of the LED monitoring and display array 32 of FIG. 4B associated with that time interval may be lit as green, for example, to indicate the monitored variable value remains below all thresholds. The monitored variable's value then rises above the first (yellow) threshold between times 1 and 2 (t₁-t₂), and remains below the second (red) threshold in the graphic 30′ of FIG. 4A, so the high speed LED associated with that time interval may be lit as yellow in FIG. 4B's LED monitoring and display array 32. During times 2 to 3 (t₂-t₃) the monitored variable's value rises above the second (red) threshold in FIG. 4A's graphic 30′ causing the high speed LED with times between 2 and 3 to be lit as red in the LED monitoring and display array 32. The monitored variable's value _{at time 4} (t₄) crosses back into the yellow threshold region in the graphic 30′ of FIG. 4A and would be lit as yellow (not shown) and finally at time 4, the monitored variable's value is back under all thresholds and would be lit as green (note shown).

Other colors may be used to achieve the objectives of the present invention, green, yellow and red are merely exemplary, illustrative and non-limiting. In addition, as discussed above, additional (or fewer) thresholds may be established. In addition, the delineation between rotational speeds may comprise more or fewer than low, mid and high speeds, including in some embodiments that have a single column of LEDs that apply to any rotational speed.

For all embodiments discussed herein, the color changing or annunciation (including without changing colors) of LEDs in an LED monitoring and display array may be “sticky” in certain embodiments in that the peak values are “remembered” and displayed in a way that annunciates or indicates to an operator that the color or lighting is a peak value. For example, a flashing LED may indicate a peak value, or an LED that remains lit when the other LEDs are not lit or actuated. In this regard, the monitored variable value data may be obtained by a speed, current, and/or voltage sensor operatively connected with the electric motor and/or rotational drive shaft and, in turn, further in operative communication with a processor which comprises programmed instructions to receive the monitored variable(s) values over the rotational atherectomy procedure and to store them, e.g., with a memory in operative communication with the processor. In this way, the peak values are retained for informing the operator through the visual annunciator(s), e.g., an LED monitoring and display array. Speed, current and/or voltage sensors and/or sensing may occur internally within the atherectomy device and/or electric motor.

In various embodiments, the LEDs of the LED monitoring and display array may be reset by the start of another procedure and/or be reset by pressing a speed switch on the handle of the device.

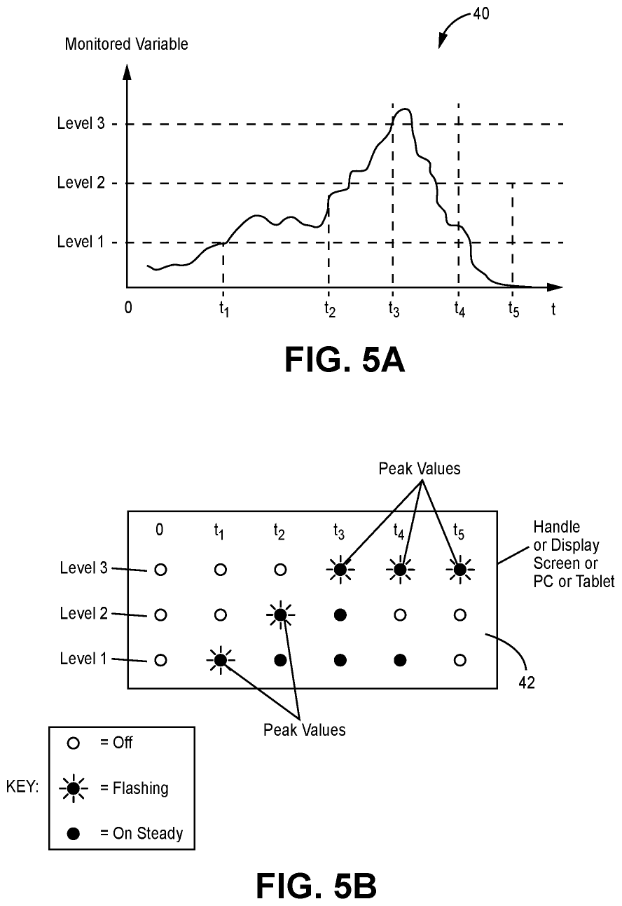

FIGS. 5A and 5B provides another exemplary embodiment illustrating graphic 40 that graphing the monitored and sensed variable over time during a rotational medical device procedure, e.g., rotational atherectomy, and another exemplary embodiment of an LED monitoring and display array 42 comprising a series of time points ranging from, for example, time 0 to time 5 (t₀ to t₅), corresponding with the graph of FIG. 5A. The LED monitoring and display array 42 may be viewed as a bar graph with each row corresponding with the three levels (thresholds) of monitored variable values ranging from lowest value to highest value as follows: a first threshold (Level 1), a second threshold (Level 2) and a third threshold (Level 3). FIGS. 5A and 5B also demonstrate annunciation of peak values and the stickiness of that annunciation. Other embodiments may not require or provide annunciation of peak values and/or stickiness of that annunciation as indicated by the sunburst effect around peak LEDs within each Level which may comprise flashing or other indicator of peak value.

Still referring to FIGS. 5A and 5B, at time 0 in graphic 40 of FIG. 5A, there is no monitored value to report or display, so all LEDs in that column are not lit.

Between times 0 and 1 (t₀-t₁), the monitored value crosses Level 1 in graphic 40, resulting in a blinking or flashing LED in the row corresponding with Level 1 threshold in the array 42 of FIG. 5B. This flashing or blinking LED indicates that a peak value has been achieved. The flashing or blinking is illustrated by the sunburst elements in the array 42, and the key to the array 42.

Between times 1 and 2 (t₁-t₂), the value crosses Level 2 in graphic 40, resulting in a blinking or flashing LED in the row corresponding with the Level 2 threshold of array 42. In addition, as shown, certain Levels may comprise LEDs that are actuated or lit, but may not be flashing in some embodiments, for example when the actuated or lit LED is not correspondent to a peak value.

At time 3 (t₃), the value crosses Level 3 of graphic 40, resulting in a blinking or flashing LED in the row corresponding with the Level 3 threshold in array 42, thereby indicating peak value. In addition, as shown, the Level 1 and Level 2 threshold LEDs may be lit solidly in array 42, but may not be flashing in some embodiments in order to indicate real time monitored variable values.

At time 4 (t₄), the value is between Level 1 and Level 2 in graphic 40, so the Level 1LED is lit, but is shown as a solid, non-flashing LED in array 42 because peak value at time 3was determined to be above Level 3. As a result, that Level 3 LED in array 42 remains flashing for the remainder of the procedure.

At time 5 (t₅) of graphic 40, the Level 3 LED in array 42 is flashing, but the real time variable value has dropped to zero, so no other time 5 (t₅) LEDs in array 42 are actuated.

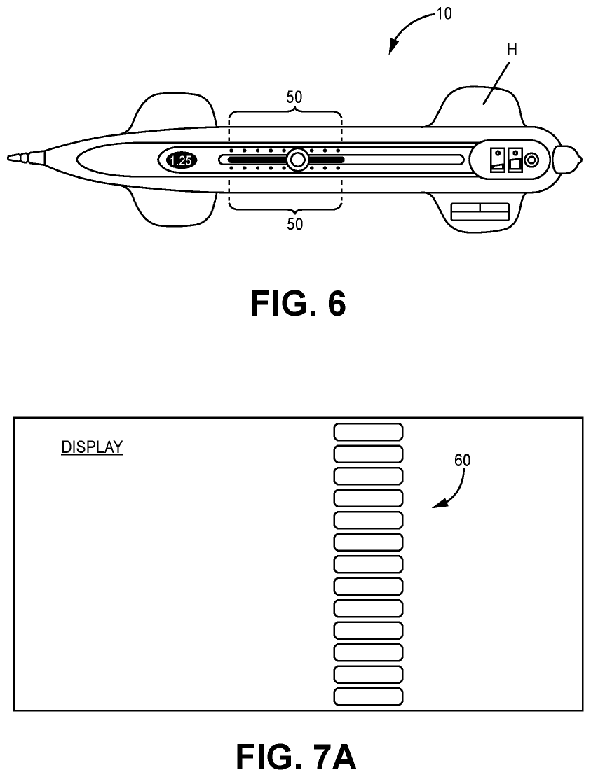

FIG. 6 illustrates one embodiment wherein the LED monitor and display for monitoring a variable is incorporated into, or on, the device handle of an exemplary rotational atherectomy device 10 that is discussed above in connection with FIGS. 1A and 1B. In the illustrated embodiment, an exemplary rotational medical device 10 such as a rotational atherectomy device, or orbital rotational atherectomy device may comprise a handle H with an LED monitor and display array 50 thereon, or incorporated therein. Other intravascular motor-driven devices with handles may also incorporate the LED monitor and display within the handle. The LED monitor and display illustrated is exemplary and may comprise one or more of the LED monitor and display embodiments described herein.

The LED monitor and display of FIG. 6 provides an LED monitoring and display array 50 comprising a single line of LEDs (or other annunciators) on both sides of the channel along which the advance knob A translates. The LEDS of array 50 are lit when the monitored variable, e.g., current, reaches a predetermined magnitude that corresponds with a given LED within the array 50. As the magnitude of the monitored variable increases, one or more LEDs in the array 50 may be lit to allow the operator visual feedback of the monitored variable's magnitude. In some embodiments, the LEDs may change color as the magnitude of the monitored variable increases and reaches predetermined thresholds, e.g., lower magnitudes may be within a lower predetermined threshold range wherein the lit LEDs of array 50 are all a first color, e.g., green. The next higher range of magnitudes may comprise LEDs in the array 50 that are a second color different from the first color, e.g., all yellow. The next higher range of magnitudes may comprise the array 50 comprising LEDs that are a third color different from the first and second colors, e.g., all red. As noted above, the highest magnitude monitored and displayed may be “sticky” in that the highest lit or actuated LED in the array 50 may remain lit or actuated to allow the operator to visualize the highest magnitude of the monitored variable during a given procedure. In other embodiments, the LEDs in array 50 may not be “sticky”. In some embodiments, the LEDs of the LED monitoring and display array 50 may be arranged in a horizontal line, while in other embodiments, the LEDS may be arranged in a vertical line.

FIG. 6 illustrates the LED monitoring and display array 50 as arranged on one or both sides of the abrasive element advance knob A as described above in connection with the exemplary prior art rotational atherectomy devices of FIGS. 1A and 1B. The embodiment of FIG. 6 is similar to that of FIG. 2 in terms of general annunciation functionality. The location of the LED monitoring and display array 50 of FIG. 6 is, however, different from that of FIG. 2, though both are located on or integrated with handle H, providing the operator with an easier visualization of both location of the advance knob A (and associated location of the abrasive element AE), and the magnitude of the monitored variable(s).

In some embodiments, a first line of the LEDs in the array 50 may correspond to a first monitored variable, while the second line of the LEDs in the array 50 may correspond to a second monitored variable.

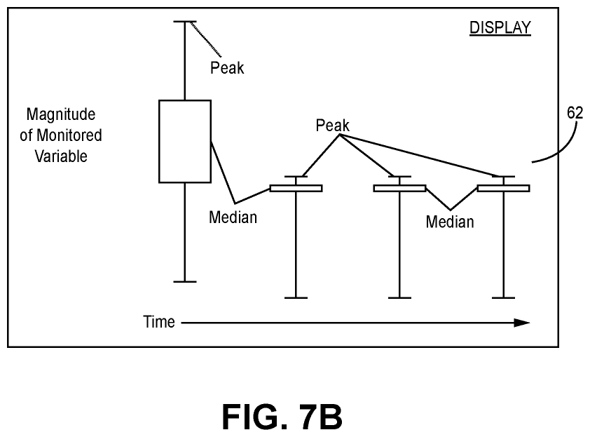

Turning now to FIGS. 7A and 7B, another embodiment of a monitoring and display array 60 for a monitored variable is provide. In some embodiments, as in FIG. 7A, a real-time monitoring and display array 60 may be provided on a display separate from the device handle H, such as a tablet or a monitor or a pc display. In other embodiments, an LED monitoring and display array 60 may be provided, as described above, as incorporated into or on an exemplary rotational medical device's handle H. FIG. 7A provides a vertical light bar graphic 60 that actuate or light and may, or may not, change colors as the magnitude of the monitored variable changes. In addition, the annunciated maximum or peak magnitude may be “sticky” as described above. FIG. 7B illustrates a displayed set of magnitudes of one, or more than one, monitored variable 62 that may correspond with the single bar graphic of FIG. 7A and that may be divided into low, mid or high (for example) levels of magnitude and wherein the peak magnitudes may be “sticky” and therefore retained on the display. In other embodiments, the columns of magnitude data on FIG. 7B may each reflect a monitored and displayed set of magnitudes for different variables that are monitored.

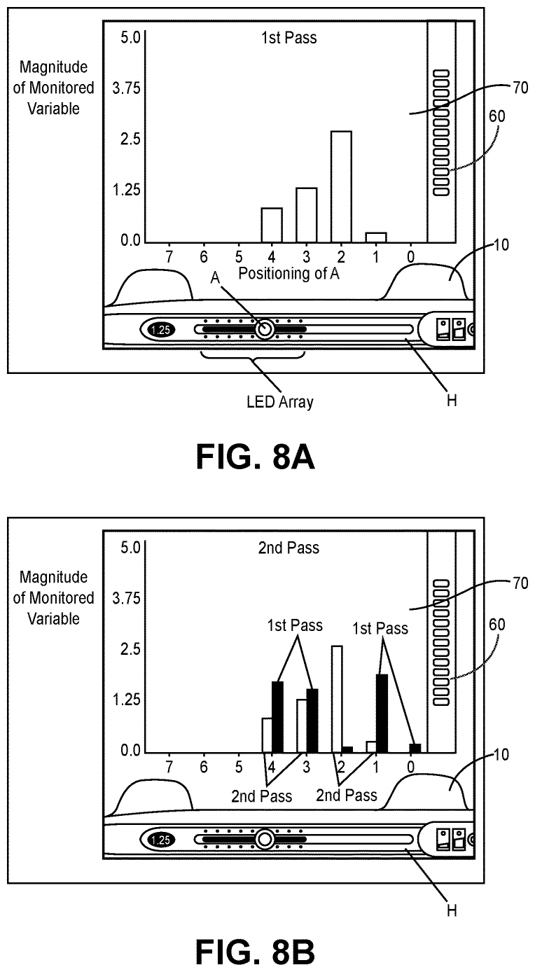

FIGS. 8A and 8B illustrate another exemplary embodiment for monitoring and displaying a variable during operation of a rotational medical device such as an atherectomy or rotational atherectomy device. Here, as shown in FIG. 8A, the magnitudes reached during a first (axial) pass of an abrasive element AE through an exemplary lesion are shown, relative to the position of the advance knob A of the exemplary rotational atherectomy device 10 as discussed in relation to FIGS. 1A and 1B. As discussed, the position of the advance knob A is correspondent to the location of the abrasive element AE within the patient's vasculature.

Thus, the X-axis of the display provides a relative position scale ranging from 0-7 correspondent to the longitudinal or axial position of the advance knob A. As the advance knob A is moved in a distal direction (away from position scale “0”), thereby causing the rotational drive shaft S and connected abrasive element AE to move distally, the magnitude of the monitored variable relative to the position of the advance knob A is captured by a relevant sensor and communicated through a processor after processing via programmed instructions to a display. As the advance knob A is moved distally to achieve more distal positions, the related magnitudes are captured and displayed according to, or categorized by, the position of the advance knob A. The advance knob A may be moved in the distal direction in a “first pass” through the lesion, whereby the measure magnitudes of the variable are captured and displayed. In addition, the advance knob A may be moved in the proximal direction to return the advance knob A, drive shaft S and abrasive element AE to a starting position of “0”, wherein the magnitudes on the proximal movement (or a second half of the first pass) are also measured, captured and displayed. The graphic display 70 of FIG. 8A provides one embodiment of the resulting annunciation display.

FIG. 8A also may include the LED array of FIG. 6 that is located along the advance knob A channel. Alternatively, or in combination with the LED array along the advance knob A channel, an LED light bar similar to the graphic 60 of FIG. 7A.

A “second pass” of the abrasive clement AE through the lesion may be desired, depending on the measured magnitudes obtained and the related procedural progress achieved with the first pass. Graphic 70 related to an exemplary second pass is provided in FIG. 8B, with the results of the first pass retained for comparison and determination of progress and/or completion of the procedure in graphic display 70. More than two passes may be required, wherein each set of measured and captured magnitude data may be displayed. Alternatively, the last two pass data sets may be displayed on graphic display 70. As with the other annunciation displays discussed herein, the measured and captured data may be displayed on a display that is in communication with the rotational medical device that is either separated from the device, e.g., a monitor, tablet and/or PC. Alternatively, the measured and captured data as in FIGS. 8A and 8B may be displayed on a screen that is integrated on or within the handle H of a rotational medical device as described herein.

In each embodiment discussed herein, one or more sensors are required to measure the monitored variable(s) magnitude as is understood in the art. The sensor(s) may be in operative communication with the motor driving the rotational medical device and a processor that is configured to provide the measured and/or captured magnitude data to a display that is in operative communication with the processor where the data is displayed as described herein. In some embodiments, the processor may comprise programmed instructions and be configured to execute the programmed instructions to provide the data in the desired format. In some embodiments, a separate memory may be in operative communication with the, wherein the programmed instructions are stored and configured to be accessed by the processor and/or wherein the memory stores the measured and/or captured data. The various sensors that may be incorporated into the inventive embodiments comprise a current sensor, a rotational speed sensor, a torque sensor, and/or a voltage sensor. In each case, the sensor monitors and captures the magnitude of the monitored variable and transmits the captured magnitude to the microcontroller or processor for processing and/or display.

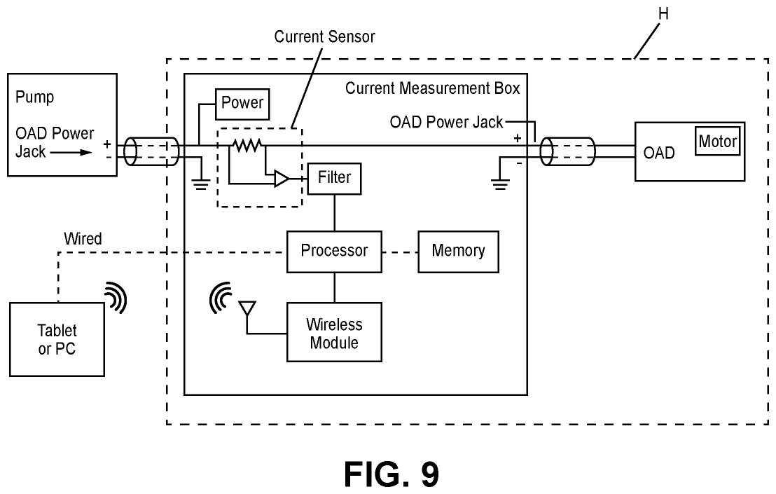

Turning now to FIG. 9, a schematic diagram of an exemplary rotational atherectomy device is provided and comprises an exemplary orbital atherectomy device (“OAD”) with handle and an exemplary in-line current sensor. Thus, an electric motor is operatively connected via a first OAD power jack with a pump that comprises a second OAD power jack. A current measurement box is electrically connected with, and disposed between, the OAD with motor and the pump as illustrated. The current draw of the OAD with motor is sensed at current sensor within the current measurement box after drawing some power for at least the sensing step, filtered at filter(s) and then transmitted to an operatively connected microcontroller or processor comprising programmed instructions, wherein a current level is determined. The determined current level is then transmitted via wireless, e. g, Bluetooth, or a wired connection to a display that may comprise a display screen and/or a PC or table as shown.

As shown, the handle H may surround the current measurement box, the motor and the OAD power jack. In the illustrated embodiment, the pump is located outside of the handle.

For all LED displays described herein, some embodiments may comprise the LED display on an external screen, e.g., a monitor, a computer screen, a tablet or the like. In other embodiments, the LED display may be incorporated into the handle of the device as described above. As shown in FIG. 9, a wireless module may be provided within the handle and in operative communication with the microcontroller or processor for wireless data transmission to a display. Alternatively, a wired connection from the microcontroller or processor to a display may be provided.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. A system for monitoring a variable of an atherectomy procedure executed by a rotational atherectomy device having a handle that surrounds an electric motor, and a rotational drive shaft in operative connection with the electric motor, the system comprising:
a sensor for sensing the value of the monitored variable;
a processor in operative communication with the sensor and comprising programmed instructions configured to determine whether the sensed value is above or below at least one threshold value;
a display operatively connected with the processor and comprising an array of LED lights, wherein the processor is further configured to actuate one or more of the LED lights to indicate whether the sensed value is above or below the at least one threshold value,
wherein the processor is further configured to annunciate one or more peak values reached during the atherectomy procedure, and
wherein the display is integrated into the handle of the rotational atherectomy device.

2. The system of claim 1, wherein the annunciated peak value is retained on the display during at least a portion of the atherectomy procedure.

3. The system of claim 1, wherein the annunciated peak value is annunciated on the display by a flashing LED within the array of LEDs.

4. The system of claim 1, wherein the annunciated peak value is annunciated by a constantly lit LED within the array of LEDs.

5. The system of claim 2, wherein the annunciated peak value is annunciated on the display by a flashing LED within the array of LEDs.

6. The system of claim 2, wherein the annunciated peak value is annunciated by a constantly lit LED within the array of LEDs.

7. The system of claim 1, wherein the array of LEDs comprises more than one color and at least one threshold value, wherein one of the more than one color annunciates a sensed value that is below the at least one threshold value and wherein a second of the more than one color annunciates a sensed value that has met or exceeded the at least one threshold value.

8. The system of claim 1, wherein the more than one color is correspondent to rotational speed thresholds.

9. The system of claim 1, wherein the array of LEDs is configured to be reset by an operator.

10.-11. (canceled)

12. The system of claim 1, wherein the monitored variable comprises one or more of the group consisting of:
a rotational speed of the electric motor and/or the rotating rotational drive shaft;
a rate of change of the rotational speed of the electric motor and/or the rotational drive shaft;
a voltage of the electric motor;
a rate of change of the voltage of the electric motor; and
a current generated by the electric motor;
a rate of change of the current generated by the electric motor during a rotational procedure.

13.-15. (canceled)

16. A rotational medical device comprising:
a prime mover;
rotational drive shaft operatively connected with the electric motor; a pump operatively connected with the prime mover; and
a current measurement box electrically connected with the prime mover and with the pump, wherein the current measurement box comprises a current sensor that is configured to sense an amount of current drawn by the prime mover during a rotational medical procedure.

17. The rotational medical device of claim 16, further comprising:
a first power jack in operative communication with the prime mover,
a second power jack in operative communication with the pump,
whereby the prime mover and the pump are electrically connected.

18. The rotational medical device of claim 17, further comprising:
the current measurement box configured to draw power before the current sensor senses the amount of current drawn by the prime mover.

19. The rotational medical device of claim 18, further comprising:
one or more filters in electrical communication with the current sensor,
a processor in electrical communication with a processor comprising programmed instructions and configured to determine the current level drawn by the prime mover.

20. The system of claim 16, wherein the motor and the current measurement box are located with a handle of the rotational medical device.

21. A rotational atherectomy system comprising:
an electric motor;
a rotational drive shaft operatively connected with the electric motor;
a pump operatively connected with the electric motor;
a current measurement box electrically connected with the electric motor and with the pump, wherein the current measurement box comprises a current sensor that is configured to sense an amount of current drawn by the electric motor during a rotational atherectomy procedure; and
a display in operative communication with a processor, wherein the processor is configured to execute programmed instructions to determine a current value and communicate the determined current value to the display, wherein the determined current value is displayed.

22. The rotational atherectomy system of claim 21, further comprising:
a first power jack in operative communication with the prime mover,
a second power jack in operative communication with the pump, whereby the electric motor and the pump are electrically connected.

23. The rotational atherectomy system of claim 22, further comprising:
the current measurement box configured to draw power before the current sensor senses the amount of current drawn by the electric motor.

24. The rotational atherectomy system of claim 23, further comprising:
one or more filters in electrical communication with the current sensor,
a processor in electrical communication with a processor comprising programmed instructions and configured to determine the current level drawn by the electric motor.

25. The rotational atherectomy system of claim 21, wherein the motor and the current measurement box are located with a handle of the rotational atherectomy device.
